(19) United States
(12) Patent Application Publication
(10) Pub. No.: US 2021/0299310 A1
(43) Pub. Date: Sep. 30, 2021

(54) COPPER BEARING SUBSTRATE FOR ODOUR CONTROL

(71) Applicant: LYNDON SMITH, ORILLIA (CA)

(72) Inventor: LYNDON SMITH, ORILLIA (CA)

(21) Appl. No.: 16/827,933

(22) Filed: Mar. 24, 2020

(51) Int. Cl. A61L 2/238; A43B 17/04

(52) U.S. Cl. CPC A61L 2/238; A43B 17/04

(57) ABSTRACT

A substrate having discrete copper laminae for positioning proximate an odour causing area of a user's body. The lamina are spaced apart to provide channels between adjacent laminae and the substrate. The high density of the laminae and channels cooperate to control odour. Several embodiments are disclosed for use in a variety of clothing and footwear articles.

### FIELD OF THE INVENTION

The present invention relates to a substrate having copper laminae thereon and more particularly, the present invention relates to a substrate to be positioned by proximate an odour causing area of a user's body to control odour.

### BACKGROUND OF THE INVENTION

Odour control has always been an issue of concern, particularly with people inclined to athletics. Generally, we live in a more active society and a panacea of athletic footwear and clothing is available with moisture wicking, body temperature control, odour checking among others.

Although such clothing and footwear is useful, unfortunately due to the materials used in fabrication, with prolonged use, the efficacy diminishes and odours can be retained despite regular laundering.

In the literature, namely an article from Infection Control Today, the benefit of copper as an antimicrobial and deodorizer are documented. In the article, Copper Fights Bacteria and Now Battles Odors, Oct. 27, 2010, Environmental Hygiene, Antibiotics & Antimicrobials, Archive, it was indicated:

“Scientists are reporting development of a new approach for dealing with offensive odors, one that doesn't simply mask odors like today's room fresheners, but eliminates them at the source. Their research found that a deodorant made from nanopartides hundreds of times smaller than peach fuzz eliminates odors up to twice as effectively as today's gold standard. A report on these next-generation odor-fighters appears in the American Chemical Society's journal, Langmuir.

Brij Moudgil and colleagues note that consumers use a wide range of materials to battle undesirable odors in clothing, on pets, in rooms, and elsewhere. Most common household air fresheners, for instance, mask odors with pleasing fragrances but do not eliminate the odors from the environment. People also apply deodorizing substances that absorb smells. These materials include activated carbon and baking soda. However, these substances tend to have only a weak ability to absorb the chemicals responsible for the odor.

The scientists describe development of a new material consisting of nanoparticles of silica (the main ingredient in beach sand) each 1/50,000th the width of a human hair coated with copper. That metal has well-established antibacterial and anti-odor properties, and the nanoparticles gave copper a greater surface area to exert its effects. Tests of the particles against ethyl mercaptan, the stuff that gives natural gas its unpleasant odor, showed that nanoparticles were up to twice as effective as the gold standard activated carbon at removing the material's foul-smelling odor. In addition to fighting odors, the particles also show promise for removing sulfur contaminants found in crude oil and for fighting harmful bacteria, they add.

Reference: Moudgil B, et al. Copper Coated Silica Nanoparticles for Odor Removal. Langmuir. 2010, 26 (20), pp 15837-15844.”

The efficacy of copper has also been recognized in the clothing industry. Kobayashi et al, in Deodorizing and Antibacterial Abilities of Knitted Cotton Fabrics Mordant Dyed with Reactive Dyes and Copper (II) Sulfate, Textile Research Journal 80(3):271-278—February 2010, discuss the following:

“Non-mercerized and mercerized knitted cotton fabrics were dyed with the copper-containing reactive dyes, C.I. Reactive Blue 237 and C.I. Reactive Blue 71, and pre- and post-mordanted with copper (II) sulfate. We investigated the effects of the type and concentration of dye, the type of cotton fabric, and the difference in mordanting methods on ethanethiol deodorizing ability. The deodorizing abilities conferred by pre-mordanting with copper sulfate (pre), dyeing (D), dyeing after pre-mordanting (pre-D) or post-mordanting with copper sulfate after dyeing (D-post) were in the order D<pre-D<D-post<pre. The deodorizing abilities of the sample fabrics with higher copper ion uptakes were not necessarily higher. The abilities of the mordant-dyed non-mercerized cotton samples were higher than those of the mordant-dyed mercerized cotton samples. The mordant-dyed non-mercerized knitted cotton samples adsorb larger amounts of copper ions directly bound to cotton during pre-mordanting. The ability of the copper ions bound to cotton and to the dye during pre- or post-mordanting is higher than that of the copper ions contained in the dye molecule. In addition, mordant-dyed cotton fabrics exhibited antibacterial activity against Staphylococcus aureus and MRSA, and thus had potential as deodorizing/antibacterial fabrics.”

The patent literature presents options with antimicrobial agents, metals and commensurate materials. An example is provided in United States Patent Application No. 2002/0146950, published Oct. 10, 2002, by Reich. The document discloses garments which polyester fibres and antimicrobial acetate fibres. The fibres are combined to provide an antimicrobial fibre used to manufacture articles of clothing.

The publication provides a useful solution, however, as noted above, such materials as polyester have a tendency to retain odour with extended use.

Oakley, in U.S. Pat. No. 4,864,740, issued Sep. 12, 1989, provides a disposable insole and a method for forming same.

The patent teaches a multiple layer hygienic insole construction. The layers include polypropylene, a composite of pulp fibres and polypropylene meltdown on the top layer and a bottom layer comprising polyethylene vinyl acetate meltdown. The layers are indicated to include antimicrobial agents, fragrance, neutralizer , etc.

The product is fibrous and as such is susceptible to fibre density variations in the overall structure. In light of this, the antimicrobial additive would also be inconsistent throughout the insole structure to provide selective efficacy.

It would be desirable to have an odour controlling substrate that does not rely on amphoteric solutions, disposition in threads or fibres, polyester or polyethylene and which could be used repeatedly over a prolonged period and further which does not require laundering.

The present invention satisfies these requirements as well as many other in an efficient manner.

### SUMMARY OF THE INVENTION

One object of the present invention is to provide an improved substrate useable in a variety of configurations for control of body odour.

A further object of one embodiment of the present invention is to provide a copper bearing substrate for controlling human body odour, comprising:

a substrate for positioning proximate an odour producing area of a user's body;
a plurality of discrete copper laminae disposed on the substrate in spaced relation, adjacent laminae forming tortuous channels defined by the adjacent laminae and the substrate, the discrete laminae having at least two common channels with adjacent laminae.

It has been found that the use of a plurality of the discrete laminae has high efficacy in terms of controlling odour. The present invention embraces the acknowledged anti-microbial action of copper and combines this with a high density arrangement of discrete laminae and channels therebetween for an effective odour controlling arrangement.

When embodied as an insole, the discrete laminae provide a texture and massaging effect to the foot of the user. In view if the fact that there is a plurality of the laminae present on the insole surface, the active copper surface area is greatly increased essentially providing multiple odour reducing zones over the insole area.

As a further beneficial effect, the channels formed by the elevation of the laminae relative to the substrate, facilitate air flow over the entire area occupied by the laminae.

This contributes to cooling and evaporation of moisture to further mitigate bacterial issues causing the odour.

As a further object of one embodiment of the present invention, there is provided a method for controlling human body odour, comprising:

providing a substrate for positioning proximate an odour producing area of a user's body, the substrate including a plurality of discrete copper laminae disposed on the substrate in spaced relation, adjacent laminae forming tortuous channels defined by the adjacent laminae and the substrate, the discrete laminae having at least two common channels with adjacent laminae; and
positioning the substrate proximate said odour producing area of a user's body to control odour.

Further use of the arrangement discussed herein can be found in various forms of, for example, luggage.

Accordingly, a further object of one embodiment of the present invention is to provide a copper bearing substrate for controlling human body odour, comprising: a substrate for positioning proximate an odour source;

a plurality of discrete copper laminae disposed on said substrate in spaced relation, adjacent laminae forming tortuous channels defined by the adjacent laminae and the substrate, the discrete laminae having at least two common channels with adjacent laminae.

Suitable examples of luggage include a suitcase, a garment bag, a sports bag, a shoe bag, a laundry bag, a clothing hamper inter alia.

In respect of alternated embodiments, it is an object of a further embodiment of the present invention to provide a copper based odour and germ controlling structure suitable for positioning adjacent a source of human body odour and/or germs, comprising:

a framework of individual copper circular or polygonal laminae connected in spaced apart relation in a predetermined shape with individual laminae being flexible relative to adjacent laminae.

The known antimicrobial feature of copper may be exploited using a networked of framework structure for the laminae to be suitable for use in prophylactic masks as an additional preventative measure.

The structure may comprise a plurality of arrays of spaced apart and connected individual copper circular or polygonal laminae. This arrangement is useful to provide some structural integrity of the copper framework so that it may be conformed to a desirable shape by a user. This is particularly convenient in the case of the mask to ensure a more precise fit which is otherwise limited by the straps and a flexible nosepiece. The open framework also contributes to effectiveness by allowing unimpeded exhalation and inhalation. To further enhance effectiveness, copper thread or powder may augment the framework arrangement.

The copper laminae may be entirely copper or copper plated base or precious metal.

Having thus generally described the invention, reference will now be made to the accompanying drawings.

### BRIEF DESCRIPTION OF THE DRAWINGS

FIG. 1 is a plan view of a first embodiment of the present invention;

FIG. 2 is an enlarged section of FIG. 2;

FIG. 3 is an alternate embodiment of FIG. 2;

FIG. 4 is an alternate embodiment of FIG. 2;

FIG. 5 is a section along line 2-2 of FIG. 2;

FIG. 6 is an exploded view of an alternate embodiment of the present invention;

FIG. 7 is a perspective view of an alternate embodiment of the present invention;

FIG. 8 is an alternate embodiment of the present invention;

FIG. 9 is an alternate embodiment of the present invention;

FIG. 10 is an alternate embodiment of the present invention;

FIG. 11 is a plan view of FIG. 10;

FIG. 12 is a plan view of a further embodiment;

FIG. 13 is a perspective view of another embodiment; and

FIG. 14 is a plan view of yet another embodiment.

Similar numerals used in the Figures denote similar elements.

### DETAILED DESCRIPTION OF THE PREFERRED EMBODIMENTS

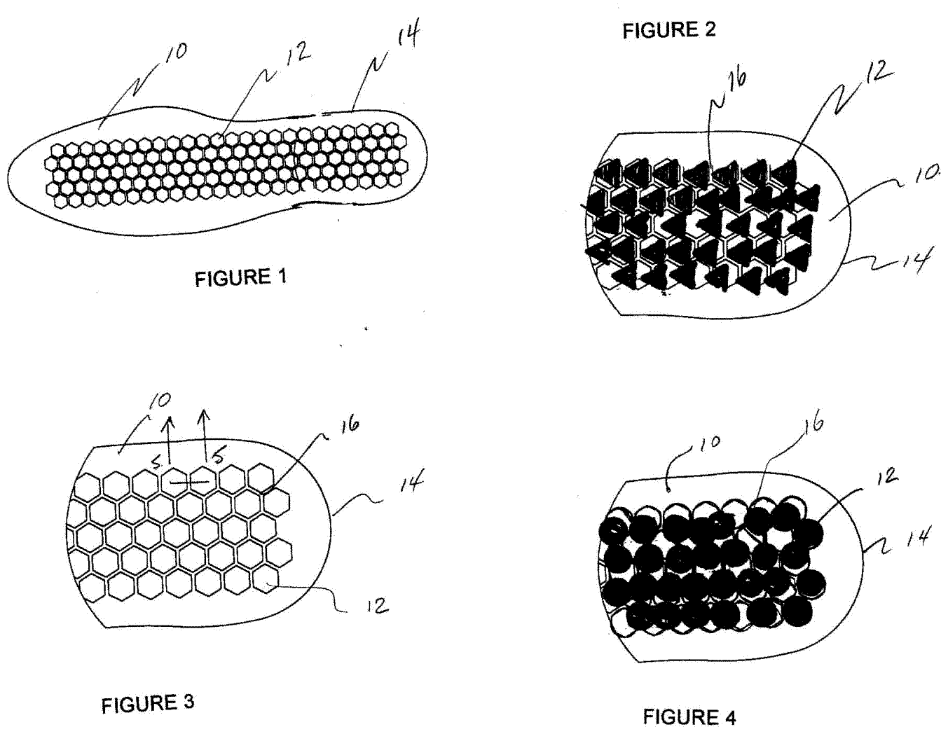

Referring to FIG. 1, shown is a plan view of one embodiment of the present invention. As illustrated, numeral 10 denotes a substrate onto which are fixedly secured individual and discrete copper lamina 12. In the example, the substrate 10 is in the shape of an insole substrate. The individual copper lamina 12 are spaced inwardly from the periphery 14 of the insole substrate 10. This facilitates customization for positioning within footwear (not shown). A useful substrate 10 which could be used in this embodiment is that created by the Zederna company, which provide cedar based insoles.

Referring now globally to FIGS. 2 through 5 and initially to FIG. 2, shown is an enlarged section of FIG. 1 and depicts the discrete copper lamina 12 as having the shape of a hexagon or honeycomb. FIGS. 3 and 4 provide alternatives in the form of a triangular lamina 12 in FIG. 3 and a circular lamina 12 in FIG. 4. In this manner, the lamina 12 may be circular, polygonal and any combination of these.

In respect of the substrate 10, it may take a number of possible forms. In the example of FIG. 1, the substrate is in the form an insole for positioning within footwear (not shown) as referenced herein previously. The insole may be composed of the materials known for such articles, i.e. a thin foam body or may be composed of a moisture wicking material, such as bamboo, cork, treated fibres, etc. The insole, in order to elevate the efficiency of the copper lamina 12, may also include copper thread therein. Securement of the copper lamina can be achieved by numerous methods depending on the specific substrate. In the case of a thin sheet substrate, a suitable adhesive resistant to heat, moisture and foot movement may be selected. The prior art provides innumerable examples. For larger substrates 10, such as an orthotic, sole of a sandal, shoe etc., the lamina 12 may be integrated into the sole material by fusion or other mechanical integration.

Further forms of the substrate 10 will be discussed herein after with the advancing Figures.

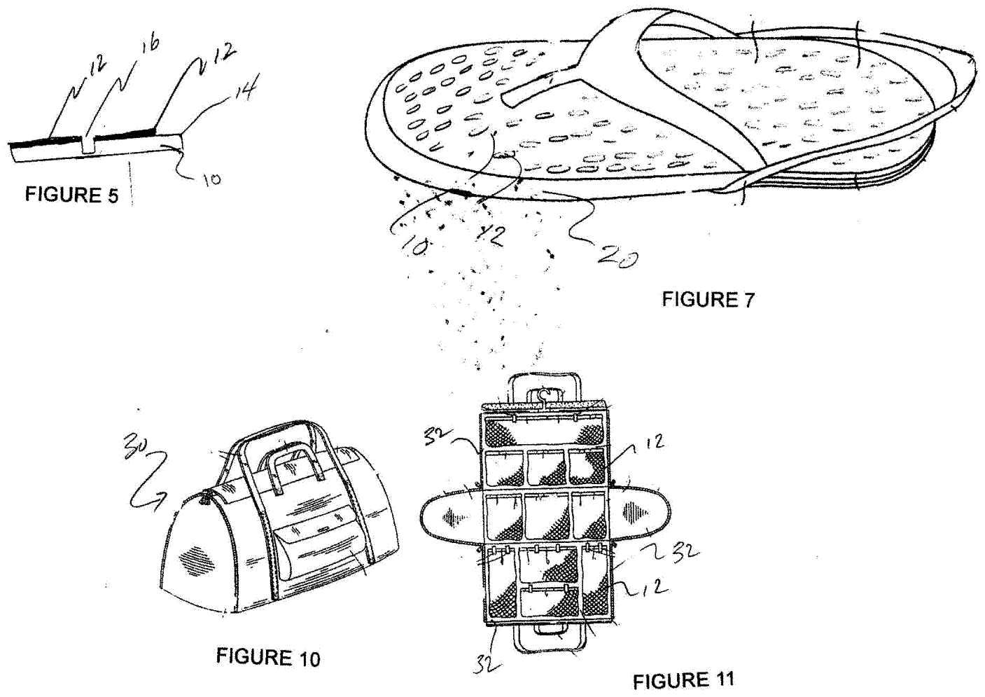

FIG. 5 is a section along line 5-5 of FIG. 2. As Illustrated, between adjacent lamina 12 there is a channel 16 defined by the adjacent lamina 12 and the substrate 10. The channels 16 are in a tortuous configuration as illustrated in FIGS. 2 though 4. Each lamina section 12 shares at least two common channels 16 with adjacent lamina 12 as illustrated in the Figures.

It has been found that the particular spacing imparts necessary flexibility and further contributes to moisture control. This is further enhanced by the fact that the channels 16 permit elevation from the substrate 10 to allow for airflow.

Spacing between lamina 12 may be from 0.001 inches (0.025 mm) to 0.500 inches (12.7 mm) and most desirably 0.070 inches (1.78 mm).

In respect of the copper lamina 12, it is desirable to provide as many laminae as possible over the area of the substrate 10. This enhances the available surface area for the copper lamina to control odour and when coupled with the tortuous channels 16, effectively provides individual odour controlling discrete zones for maximum effectiveness and minimal intrusiveness.

The dimensions of the lamina may be, for thickness between 0.001 inches (0.025 mm) and 0.06 inches (1.52 mm) and most preferably 0.012 inches (0.3 mm). In respect of the width, this will vary depending on the specific shape of the laminae.

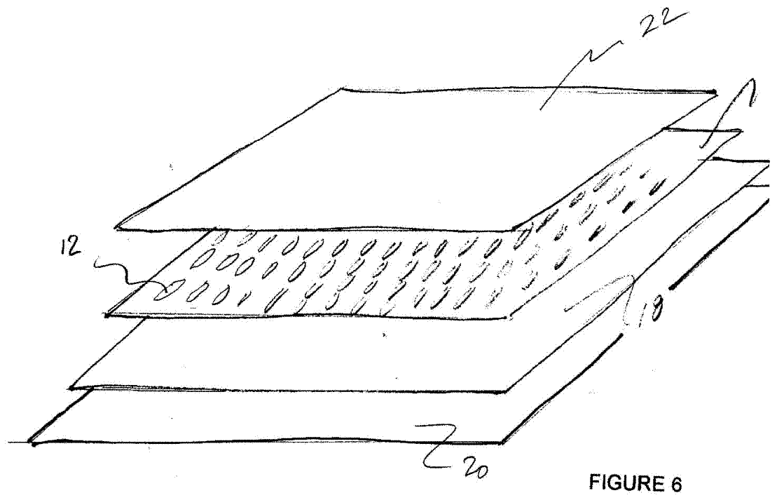

Referring now to FIG. 6, shown is an exploded illustration of an alternate embodiment.

In the example, the laminae 12 bearing substrate may include an adhesive layer 18 for fixing the substrate 10 to a secondary substrate 20, generically illustrated, examples of which will be discussed herein after.

Depending upon the specific embodiment, the arrangement may also include a top layer 22 overlying the laminae 12 and substrate 10. This layer will comprise a moisture permeable material to facilitate indirect contact of the laminae 12 with a surface of an odour susceptible part of a user's body (not shown). The layer 22 may include an adhesive for securing in a desired position.

Turning to FIG. 7, in this embodiment, the secondary substrate 20 may comprise footwear, shown in the example as a sandal. The overlying layer 22 may be present.

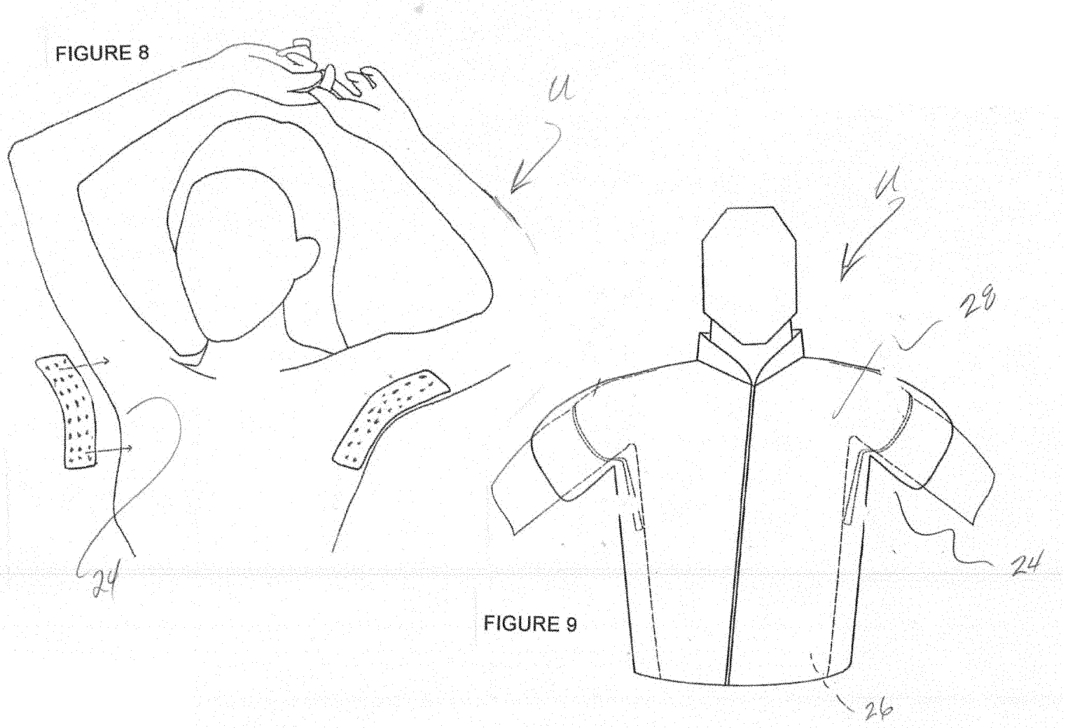

FIG. 8 illustrates yet another embodiment of the present invention where the arrangement includes a suitable bioadhesive on, for example layer 22, referenced in FIG. 6. In this example, a user, U, directly applies the arrangement shown in FIG. 6 absent substrate 20, directly to the underarm area 24 for odour control.

FIG. 9 illustrates yet another embodiment of the present invention. An undergarment 26 is provided in the form a shirt. The undergarment 26 includes a flexible substrate with the copper laminae secured to the underarm area 24. The arrangement secured takes the form similar to that illustrated in FIG. 8. Securement may be achieved by any suitable means such as stitching, adhesive etc. In this embodiment, the user, U, has an overgarment 28. The overgarment 28 and the undergarment 26 may be suitable connected to be wearable as a single or unified article of clothing.

FIGS. 10 and 11 illustrate a still further embodiment of the present invention. In this embodiment, a sports bag 30, shown in an open state in FIG. 11 includes the copper lamina 12 applied to the interior surfaces 32 of the sports bag 30. This is particularly beneficial for use with, for example, a hockey equipment or ski boot bag where the sports equipment (not shown) is often closed into the bag while still having very high perspiration content.

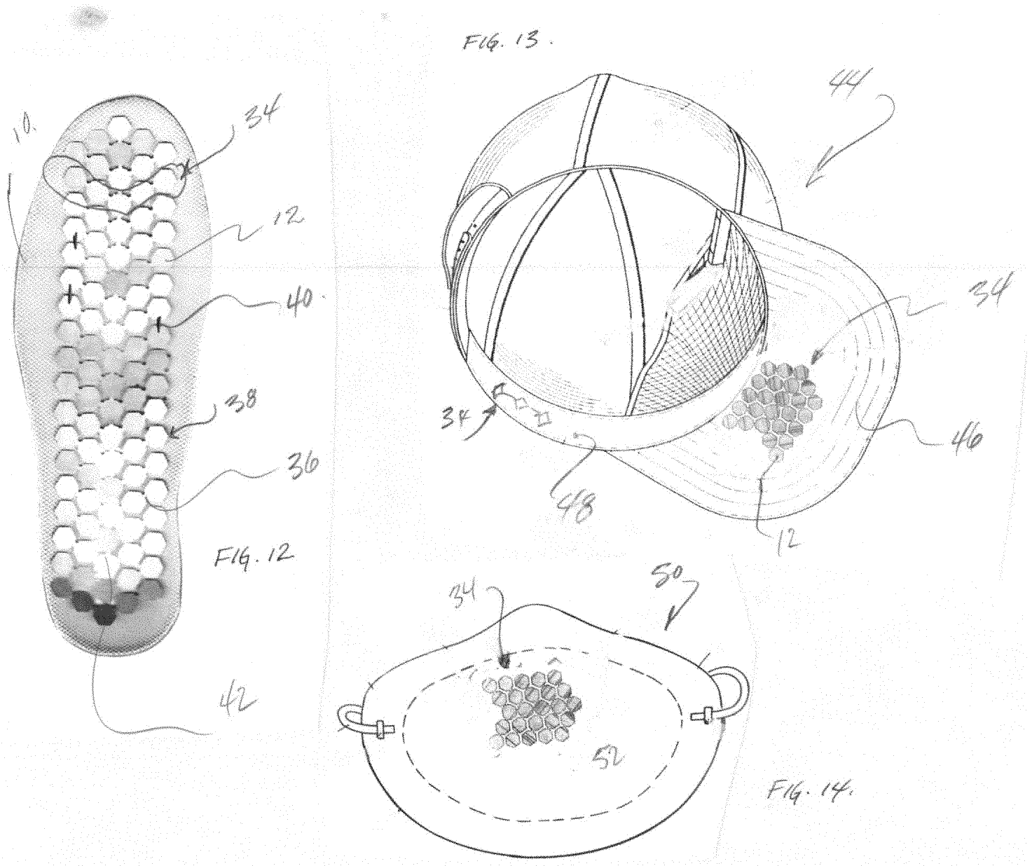

Turning now to FIG. 12, shown is a further embodiment of the present invention. In this embodiment, the laminae 12 are disposed in arrays 34 composed of individual laminae 12 connected in a predetermined formation to an adjacent laminae 12 by copper based webbing 36. Each array 34 includes a laminae having a free end 38, however, as an option, some of the terminal laminae may be connected in a predetermined arrangement indicted by numeral 40 with the webbing 36 or other suitable material. The individual connections of the laminae allows the overall structure to remain flexible and conformable which is important for the application in the insole example as shown as substrate 10.

In the example, the arrays 34 extend from a central branch 42 in a general chevron pattern. Other patterns will be possible, depending upon the specific environment in which the structure is used. The structure will be affixed to the substrate with a suitable fastening arrangement.

FIG. 13 illustrates a further embodiment where the laminae 12 arrangement discussed in FIG. 12 is positioned on a baseball cap 44. In this arrangement, the underside of the bill 46 of cap 44 may include the arrangement. The headband 48 may also include the arrays 34 either exteriorly or sewn into the headband 48.

FIG. 14 illustrates yet another embodiment where the arrays 34 are incorporated with a prophylactic mask 50. This may be done by direct fixture to the mouth area 52 of mask 50 or the structure may be inserted into a pocket 54 associated with the mask 50 shown with the dashed lines in the Figure.

Owing to the spacing of the individual lamina, breathing is not impeded in use. Further with the webbing 36, the arrangement may be bent into a comfortable and protective position in use.

It will be understood that the structure illustrated in respect of FIGS. 12 through 14 may be used in those arrangements shown in the initially referenced Figures.

As is illustrated in all embodiments, the structure is shown as a single structure, however, it will be understood that the same can be positioned on the substrate 10 in a plurality of sections.

It will be appreciated by those skilled in the art that the odour controlling arrangement set forth herein may be applied to any article of clothing, footwear, headwear and safety equipment to ameliorate odour.

I claim:

1. A copper bearing substrate for controlling human body odour, comprising:
a substrate for positioning proximate an odour producing area of a user's body;
a plurality of discrete copper lamina disposed on said substrate in spaced relation, adjacent lamina forming tortuous channels defined by the adjacent lamina and said substrate, said discrete lamina having at least two common channels with adjacent lamina.

2. The substrate as set forth in claim 1, wherein said substrate comprises an insole.

3. The substrate as set forth in claim 1, wherein said substrate comprises an orthotic.

4. The substrate as set forth in claim 1, wherein said substrate comprises an article of clothing.

5. The substrate as set forth in claim 1, wherein said substrate comprises an adhesive strip adapted for contact with a material contacting said odour producing area of a user's body.

6. The substrate as set forth in claim 1, wherein said insole comprises a moisture absorbing material.

7. The substrate as set forth in claim 6, wherein said insole includes a further copper material.

8. The substrate as set forth in claim 7, wherein said further copper material comprises at least one of copper thread and copper powder.

9. The substrate as set forth in claim 1, wherein said copper lamina comprise circular lamina.

10. The substrate as set forth in claim 1, wherein said copper lamina comprise polygonal lamina.

11. A copper bearing substrate for controlling human body odour, comprising:
a substrate for positioning proximate an odour source;
a plurality of discrete copper laminae disposed on said substrate in spaced relation, adjacent laminae forming tortuous channels defined by the adjacent laminae and said substrate, said discrete laminae having at least two common channels with adjacent laminae.

12. The substrate as set forth in claim 11, in combination with an article of luggage.

13. The substrate as set forth in claim 12, wherein said luggage is selected from the group comprising a suitcase, a garment bag, a sports bag, a shoe bag, a laundry bag and a clothing hamper.

14. A copper based odour and germ controlling structure suitable for positioning adjacent a source of human body odour and/or germs, comprising:
a framework of individual copper circular or polygonal laminae connected in spaced apart relation in a predetermined shape with individual laminae being flexible relative to adjacent laminae.

15. The structure as set forth in claim 14, wherein said frame work comprises a plurality of arrays of spaced apart and connected individual copper circular or polygonal laminae.

16. The structure as set forth in claim 15, wherein each array is connected to an adjacent array in spaced relation.

17. The structure as set forth in claim 15, wherein said framework includes a central branch from which said arrays extend.

18. The structure as set forth in claim 14, wherein said arrays have a terminal free end.

19. The structure as set forth in claim 18, wherein at least some of the terminal free ends of said arrays are connected in a predetermined pattern.

20. The structure as set forth in claim 14, wherein said individual copper circular or polygonal laminae are connected with copper based webbing.
